(19) United States
(12) Patent Application Publication
(10) Pub. No.: US 2006/0263358 A1
(43) Pub. Date: Nov. 23, 2006

(54) Method for reducing sepsis or cardiogenic shock associated with myocardial injury

(75) Inventors: Thomas Gerard Todaro, Loveland, OH (US); Kevin John Malloy, Glendale, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company, Cincinnati, OH (US)

(21) Appl. No.: 11/436,440

(22) Filed: May 18, 2006

Related U.S. Application Data
(60) Provisional application No. 60/682,638, filed on May 19, 2005.

(51) Int. Cl. A61K 39/395

(52) U.S. Cl. 424/144.1

(57) ABSTRACT

Methods of reducing sepsis or cardiogenic shock in patients experiencing myocardial injury are described. More specifically, this disclosure relates to the administration of a C5-C9 terminal complement inhibitor to patients who have had coronary artery bypass grafting or an acute myocardial infarction thereby reducing the incidence of sepsis.

### CROSS REFERENCE TO RELATED APPLICATION

This application claims priority under Title 35, United States Code 119(e) from U.S. Provisional Application Ser. No. 60/682,638, filed May 19, 2005.

### FIELD OF THE INVENTION

This disclosure relates to methods of using terminal complement inhibitor compounds in reducing the incidence of sepsis and cardiogenic shock. More specifically, this disclosure relates to the administration of pexelizumab (h5G1.1-scFv) over a short period of time to reduce the occurrence of sepsis or cardiogenic shock in patients who have had organ damage, including an acute cardiovascular event such as coronary artery bypass grafting, or an acute myocardial infarction.

### BACKGROUND OF THE INVENTION

For certain patients, coronary artery bypass grafting (CABG) is the preferred form of treatment to relieve symptoms and often increase life expectancy in those suffering from coronary artery disease. CABG consists of direct anastomosis of a vessel segment to one or more of the coronary arteries. During on pump CABG surgery, the heart is usually stopped from beating, to facilitate the anastomosis procedures. While the heart is not beating, extracorporeal circulation of the blood supports most of the patient's body (excluding the heart and, to some extent, the lungs). A cardiopulmonary bypass (CPB) machine receives deoxygenated blood from the patient's body, adds oxygen and various nutrients to the blood, and pumps the oxygenated blood back into the circulation.

Although CABG surgery has substantially improved the therapeutic outcome of patients with advanced myocardial ischemia, the recovery period may often be traumatic to the patient with significant attendant risks. For example, it is known that the CPB elicits a systemic inflammatory response that causes tissue injury and contributes to significant post-operative and long-term clinical morbidity. During CPB, exposure of blood to bioincompatible surfaces of the extracorporeal circuit, as well as tissue ischemia and reperfusion associated with the procedure induces the activation of several major humoral pathways of inflammation. Clinical manifestations attributed to this systemic inflammatory response may include myocardial injury which may manifest as myocardial infarction (heart cell death) or as severe ventricular dysfunction requiring circulatory assist. The inflammatory response that occurs during cardiopulmonary bypass has been referred to as a systemic inflammatory response syndrome (SIRS) which may have a clinical manifestation similar to that of sepsis (Ann. Thorac. Surgery 2003; 75: S715-20).

Cardiogenic shock is the leading cause of death for patients with acute myocardial infarction (AMI) who reach the hospital alive. Clinically evident SIRS is often present in patients with cardiogenic shock (Circulation 2003; 107: 2998-3002). This SIRS may play an important role in the genesis of cardiogenic shock and the outcome.

It has been generally accepted that sepsis involves a systemic inflammatory response (Lancet 2005; 365: 63-78). The complement cascade has been implicated in the pathogenesis of sepsis (Current Drug Targets—Inflammation & Allergy 2004; 3: 87-96) and systemic inflammatory response syndrome (Ann. Thorac. Surgery 2003; 75: S715-20). In humans with sepsis, there is well established evidence for the appearance of complement activation products in plasma (J. of Immunology 2001; 166: 1193-99). A systemic inflammatory response mediated at least in part by complement activation is also implicated in the genesis of cardiogenic shock (Circulation 2003; 107: 2998-3002).

Inhibition of the complement cascade has been theorized as an intervention in sepsis and cardiogenic shock. Much attention has been focused on the role of C3. The role of the terminal complement cascade, that is C5 through C9, as an intervention in sepsis is not well understood. The preferred point of intervention is unclear for sepsis (Crit Care Med 2003; 31: S97-104) and in cardiogenic shock (Circulation 2003; 107: 2998-3002). In addition, the treatment regimen for sepsis or cardiogenic shock with a C5 through C9 inhibitor of the terminal complement cascade has not been established.

Fitch et al., Pharmacology and Biological Efficacy of a Recombinant, Humanized, Single-Chain Antibody C5 Complement Inhibitor in Patients Undergoing Coronary Artery Bypass Graft Surgery With Cardiopulmonary Bypass (Circulation, 1999; 100:2499.), disclose that h5G1.1-scFv (Pexelizumab), a recombinant single-chain antibody C5 inhibitor, proved to be a potent inhibitor of systemic complement activation, inhibiting both the generation of the proinflammatory activation product C5a and C5b-9 in patients undergoing CPB. Fitch et al. further disclose that the potent complement inhibitory and terminal complement inhibitor activities of h5G1.1-scFv were associated with significant reductions in postoperative CK-MB release, new cognitive deficits, and blood loss. The potent inhibitory and terminal complement inhibitor effects of h5G1.1-scFv were associated with significant reductions in postoperative myocardial injury. In addition, Fitch measured CD11b on activated neutrophils and monocytes, and reported that in doses sufficient to completely block hemolytic activity and soluble C5b-9 generation (e.g. 1.0 and 2.0 mg/kg), h5G1.1-scFv significantly attenuated peak leukocyte CD11b expression compared with the placebo.

No short term method of treating sepsis or cardiogenic shock due to organ damage in patients such as those patients that have undergone CABG or have had an acute myocardial infarction exists in the art. Further, the relative utility of Pexelizumab (h5G1.1-scFv) to reduce sepsis is not known.

It would be advantageous to provide a method of treating or preventing sepsis in patients who have experienced organ damage by administering Pexelizumab (h5G1.1-scFv).

### SUMMARY OF THE INVENTION

A method of using a C5-C9 terminal complement inhibitor in reducing the incidence of sepsis or cardiogenic shock after organ damage has now surprisingly been found. This method includes administering a C5 through C9 terminal complement inhibitor compound to a subject as soon as practicable after the organ damage for a period of at least about forty-eight (48) hours.

In another embodiment a method of preventing or treating sepsis or cardiogenic shock is disclosed wherein a patient is administered a bolus quantity of pexelizumab prior to (i.e. during anesthesia) or during CABG surgery immediately followed by an infusion of pexelizumab for a period of time not greater than about forty-eight (48) hours.

In another embodiment a method of preventing or treating sepsis or cardiogenic shock is disclosed wherein a patient who has suffered an AMI is administered a bolus quantity of pexelizumab prior to, during or immediately after the administration of a thrombolytic agent or a primary percutaneous coronary intervention, immediately followed by an infusion of pexelizumab for a period of time not greater than about forty-eight (48) hours.

### DETAILED DESCRIPTION OF PREFERRED EMBODIMENTS

The term “myocardial injury” or “organ damage” as used herein, means an acute cardiovascular event, including but not limited to CABG, heart transplant, AMI and stroke.

The term “bolus” as used herein, means a single dose of drug usually injected into a blood vessel over a short period of time.

The terms “reducing,” “treating” or “preventing” as used herein, means that the incidence of sepsis is less likely to occur in a patient population.

The term “sepsis” as used herein, means potentially life-threatening systemic infection that can arise from infections throughout the body, including infections in the blood, lungs, abdomen, and urinary tract, etc. It may precede or coincide with infections of the bone (osteomyelitis), central nervous system (meningitis), or other tissues. Sepsis can rapidly lead to shock, adrenal collapse, and disseminated intravascular coagulopathy (a life threatening bleeding condition) and death. Sepsis can begin with spiking fevers and chills, rapid breathing and heart rate, the outward appearance of being seriously ill. These symptoms can rapidly progress to shock with decreased body temperature (hypothermia), decreased blood pressure, confusion or other changes in mental status, and blood-clotting abnormalities.

The term “cardiogenic shock” as used herein, means hypotension of <90 mm Hg systolic blood pressure lasting for at least 1 hour, not responsive to fluid resuscitation and/or heart rate correction, felt to be secondary to cardiac dysfunction, and associated with at least one of the following signs of hypoperfusion: cool, clammy skin or oliguria or altered sensorium or low cardiac index. It may be caused by extensive myocardial and other vital organ damage. New evidence suggests, that a systemic inflammatory response, complement activation, release of inflammatory cytokines, expression of inducible nitric oxide (NO) synthase (iNOS), and inappropriate vasodilation may play an important role not only in the genesis of shock but also in outcome after shock. (Ref: Hochman et al Circulation 2003;107:2998-3002)

The term “primary percutaneous coronary intervention” means such procedures as angioplasty with or without a stent and/or balloon.

The term “C5-C9 terminal complement inhibitor” as used herein means a compound that achieves its immune defensive functions by interacting with a series of intricate but precise enzymatic cleavage and membrane binding events. The resulting complement cascade leads to the production of products with opsonic, immunoregulatory, and lytic functions. A concise summary of the biologic activities associated with complement activation is provided, for example, In The Merck Manual, 16.sup.th Edition.

The complement cascade progresses via the classical pathway or the alternative pathway. These pathways share many components, and while they differ in their initial steps, they converge and share the same “terminal complement” components (C5 through C9) responsible for the activation and destruction of target cells. The classical complement pathway is typically initiated by antibody recognition of and binding to an antigenic site on a target cell. The alternative pathway is usually antibody independent, and can be initiated by certain molecules on pathogen surfaces. Additionally, the lectin pathway is typically initiated with binding of mannose-binding lectin (MBL) to high mannose substrates. These pathways converge at the point where complement component C3 is cleaved by an active protease (which is different in each pathway) to yield C3a and C3b. Other pathways activating complement attack can act later in the sequence of events leading to various aspects of complement function.

Suitable compounds for use herein are antibodies that reduce, directly or indirectly, the conversion of complement component C5 into complement components C5a and C5b. One class of useful antibodies are those having at least one antibody-antigen binding site and exhibiting specific binding to human complement component C5, wherein the specific binding is targeted to the alpha chain of human complement component C5. Such an antibody 1) inhibits complement activation in a human body fluid; 2) inhibits the binding of purified human complement component C5 to either human complement component C3 or human complement component C4; and 3) does not specifically bind to the human complement activation product for C5a. Particularly useful complement inhibitors are compounds which reduce the generation of C5a and/or C5b-9 by greater than about 30%. A particularly useful anti-C5 antibody is h5G1.1-scFv, also known as Pexelizumab. Methods for the preparation of h5G1.1-scFv are described in U.S. patent application Ser. No. 08/487,283 filed Jun. 7, 1995 now U.S. Pat. No 6,355,245 and “Inhibition of Complement Activity by Humanized Anti-C5 Antibody and Single Chain Fv”, Thomas et al., Molecular Immunology, Vol. 33, No. 17/18, pages 1389-1401, 1996, the disclosures of which are incorporated herein in their entirety by this reference.

The route of administration of the terminal complement inhibitor compound of this invention is in accord with known methods, e.g., injection or infusion by intravenous, intraperitoneal, intracerebral, intramuscular, subcutaneous, intraocular, intraarterial, intrathecal, inhalation or intralesional routes, topical or by sustained release systems as noted below. The terminal complement inhibitor compound is preferably administered continuously by infusion and/or by bolus injection. One may administer the terminal complement inhibitor compounds in a local or systemic manner.

The terminal complement inhibitor compound of this invention may be prepared in a mixture with a pharmaceutically acceptable carrier. Techniques for formulation and administration of the compounds of the instant application may be found in “Remington's Pharmaceutical Sciences,” Mack Publishing Co., Easton, Pa., latest edition. When administered systematically, the therapeutic composition should be sterile, pyrogen-free and in a parenterally acceptable solution having due regard for pH, isotonicity, and stability. These conditions are known to those skilled in the art.

Dosage formulations of the terminal complement inhibitor compound of this invention are prepared for storage or administration by mixing the compound having the desired degree of purity with physiologically acceptable carriers, excipients, or stabilizers. Such materials are non-toxic to the recipients at the dosages and concentrations employed, and may include buffers such as TRIS HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethyleneglycol. When used for in vivo administration, the terminal complement inhibitor formulation must be sterile and can be formulated according to conventional pharmaceutical practice.

The dosage employed of the terminal complement inhibitor compound of this invention will depend on a number of factors, including, but not limited to the specific terminal complement inhibitor compound to be administered. Toxicity and therapeutic efficacy of the terminal complement inhibitor molecules described herein can be determined by standard pharmaceutical procedures. The data obtained from these procedures and animal studies can be used in formulating a range of dosages for use in human. The dosage of such molecules lies preferably within a range of circulating concentrations with little or no toxicity. The dosage may vary within this range depending upon the dosage form employed and the route of administration utilized. The exact formulation, route of administration and dosage can be chosen by the individual physician in view of the patient's condition. (See e.g., Fingi et al., 1975, in “The Pharmacological Basis of Therapeutics”, Ch. 1 p. 1). A typical daily dosage might range from about 0.001 mg/kg to about 1000 mg/kg, more preferably about 0.01 mg to 100 mg/kg, more preferably about 0.050 to 20 mg/kg of the terminal complement inhibitor compound might be an initial candidate dosage for administration to the patient. In one embodiment of the invention the daily dosage of Pexelizumab is about 3.2 mg/kg.

Using the methods in accordance with this disclosure, sepsis or cardiogenic shock in patients having a myocardial injury can be reduced. The method comprises the administration of a C5-C9 terminal complement inhibitor to a patient after suffering from such an injury.

The methods of determining effectiveness of a C5-C9 terminal complement inhibitor compound, in particular an agent that specifically blocks the activation of complement at the level of CS in reducing the incidence of sepsis in accordance with this disclosure includes the step of administering such a terminal complement inhibitor compound to a subject group including one or more patients undergoing a procedure which involves cardiopulmonary bypass. Such procedures include, but are not limited to CABG and heart transplant.

The methods of determining effectiveness of an terminal complement inhibitor compound in reducing the incidence of cardiogenic shock in accordance with this disclosure includes the step of administering a terminal complement inhibitor compound prior to or with a thrombolytic agent or a primary percutaneous coronary intervention to a subject group including one or more patients who have suffered a cardiovascular event such as an AMI.

In particularly useful embodiments, a first bolus dose of the C5-C9 terminal complement inhibitor compound is administered prior to, including but not limited to during the administration of anesthesia or during CABG surgery for a short period of time followed by a steady, continuous infusion of a second dose of the terminal complement inhibitor compound over a period of time of not more than 48 hours. In the case of a cardiovascular event such as acute myocardial infarction, the first bolus dose, should be administered as soon as possible after the event or symptoms or prior to or during the administration of a thrombolytic agent or a primary percutaneous coronary intervention, followed by a steady, continuous infusion of a second dose of the terminal complement inhibitor compound over a period of time of not more than 48 hours. In either case, the infusion of the second continuous dose of the terminal complement inhibitor compound preferably begins no later than 4 hours after the first dose. The infusion of the second continuous dose of the terminal complement inhibitor compound should be administered over a period of at least about 4 hours, preferably from about 8 to about 48 hours, more preferably, over a period of from about 12 to about 24 hours. However, it should be understood that other dosage regimes may also be useful.

All documents cited are, in relevant part, incorporated herein by reference; the citation of any document is not to be construed as an admission that it is prior art with respect to the present invention.

The following non-limiting examples are included to illustrate the present invention but are not intended to limit the scope thereof.

### EXAMPLE 1

A suitable drug product for administration to patients is a sterile, pH buffered, isotonic formulation of the pexelizumab (h5G1.1-scFv) antibody.

The following formulation is prepared

| | Formulation | Quantity per | |
|---|---|---|---|
| Ingredients | Concentration | 50 mL Vial | |
| Pexelizumab | 2.0 mg/mL | 100 | mg |
| Sodium acetate | 16.7 mM | 114 | mg |
| | (2.27 g/L) | | |
| Glacial acetic acid | 3.3 mM | 9.5 | μL |
| | (0.19 mL/L) | | |
| Sodium chloride | 150 mM | 439 | mg |
| | (8.77 g/L) | | |
| EDTA | 1 mM | 19 | mg |
| | (0.37 g/L) | | |
| Polysorbate 20 | 0.02% | 10 | mg |
| | (0.2 g/L) | | |

| | | |
|---|---|---|
| Water for injection | N/A | Q.S. |

This liquid formulation of Pexelizumab is sterile filled into 50 mL Type I borosilicate glass vials. The vials are stoppered with rubber stoppers and sealed with aluminum and polypropylene flip seals. The formulation may be administered by infusion after diluting into IV solutions such as normal saline, half-normal saline, or 5% dextrose in water (D5W) or non-diluted if administered for periods of less than 10 minutes.

### EXAMPLE 2

A Phase II Randomized, Parallel, Double-Blind, Multicenter, Placebo-controlled Study of the Effect of Pexelizumab on All-Cause Mortality and Myocardial Infarction if Patients Undergoing Coronary Artery Bypass Graft (CABG) Surgery with Cardiopulmonary Bypass

A randomized, multi-center, double-blind, placebo-controlled study was conducted of h5G1.1-scFv (Pexelizumab) administered to patients at moderately increased risk of adverse post-operative ischemic events undergoing CPB as part CABG.

The study population consisted of individuals who elected to undergo non-emergent coronary-artery bypass graft (CABG) surgery, with or without valve surgery, which required the use of a cardiopulmonary bypass (CPB) machine. There were about 3099 patients enrolled.

Patients were randomized to receive one of the following treatments: i) Bolus 2.0 mg/kg h5G1.1-scFv within about 10 minutes of being anesthetized or undergoing the procedure followed by 0.05 mg/kg/hr h5G1.1-scFv for 24 hours or ii) placebo. The h5G1.1-scFv or matching placebo was provided as a solution for injection.

Patients were seen 14, 30, 90, and 180 days after CABG surgery.

A significant decrease in the incidence of sepsis was provided by h5G1.1-scFv. At the 90 day observation point only 1.9% of the h5G1.1-scFv patients (n=1503) had septicemia compared to 3.1% of the placebo patients (n=1487) (p=0.03).

### EXAMPLE 3

Over 4254 patients enrolled at sites; 2112 were randomized to placebo and 2142 were randomized to pexelizumab. Within each treatment group, the majority (>83%) of patients underwent CABG without valve surgery. More than 59% of the patients in each treatment group were male and more than 40% were in the under 65 age category.

Patients were screened prior to their bypass surgery. There was a 24-hour treatment period, followed by a 30-day primary observation period and telephone follow-up at days 90 and 180.

Study medication was administered to patients with patients to receive 1 of 2 treatment combinations:
a) Placebo group—patients received a 10-minute bolus of placebo as soon as possible following the induction of anesthesia, but no later than 10 minutes prior to CABG, immediately followed by a 24-hour infusion of placebo; and
b) Pexelizumab group—patients received a 10-minute bolus of the formulation of Example 1 with the patient receiving 2.0 mg/kg pexelizumab as soon as possible following the induction of anesthesia, but no later than 10 minutes prior to CABG, immediately followed by a 24-hour infusion of 0.05 mg/kg/hr.

At all time points post-CABG, the pexelizumab group had fewer patients with sepsis compared with placebo. At Day 30 the incidence of sepsis was 3.1% in placebo and 2.1% in pexelizumab (p=0.05); at Day 90 the incidence of sepsis was 4.1% in placebo and 2.9% in pexelizumab (p=0.03); at Day 180 incidence of sepsis was 4.5% in placebo and 3.0% in pexelizumab (p=0.01).

### EXAMPLE 4

Patients presenting within 6 hours of the onset of symptoms of AMI will receive 2 mg/kg IV bolus pexelizumab over 10 minutes, followed as soon as possible by 0.05 mg/kg per hour infusion of Pexelizumab at a continuous IV drip rate of 20 mL/h over the next 24 hours. The entire bolus of Pexelizumab is given before balloon inflation and/or stent placement. Patients given pexelizumab will have fewer occurrences of sepsis.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

What is claimed is:

1. A method of preventing sepsis or cardiogenic shock in a subject experiencing myocardial injury comprising: administering a safe and effective amount of a C5-C9 terminal complement inhibitor compound as soon as possible prior to, after or a combination of prior to and after such injury.

2. A method as in claim 1 wherein the terminal complement inhibitor compound is an antibody that directly or indirectly reduces the conversion of complement component C5 into complement components C5a and C5b.

3. A method as in claim 2 wherein the antibody is an antibody comprising at least one antibody-antigen binding site, said antibody exhibiting specific binding to human complement component C5, said specific binding being targeted to the alpha chain of human complement component C5, wherein the antibody 1) inhibits complement activation in a human body fluid; 2) inhibits the binding of purified human complement component C5 to either human complement component C3 or human complement component C4; and 3) does not specifically bind to the human complement activation product for C5a.

4. A method as in claim 2 wherein the complement inhibitor specifically binds to a component forming the C5b-9 complex.

5. A method as in claim 4 wherein the complement inhibitor is h5G1.1-scFv.

6. A method as in claim 5 wherein the complement inhibitor is administered in two doses.

7. A method as in claim 6 wherein the two doses comprises a first bolus dose and a second continuous dose administered over a period of not more than 48 hours.

8. A method of reducing sepsis or cardiogenic shock in subjects having coronary artery bypass graft or an acute myocardial infarction comprising: administering to the subject a first bolus dosage of terminal complement inhibitor compound about ten minutes prior to the graft or anesthesia or not more than twenty four hours after symptoms of the acute myocardial infarction; and subsequently administering a second continuous dose of the terminal complement inhibitor intravenously over a period of not more than about 48 hours.

9. A method as in claim 8 wherein the second dose is administered over a period of about 8 to about 48 hours.

10. A method as in claim 9 wherein the second dose is administered over a period of about 12 to about 24 hours.

11. A method as in claim 10 wherein the second continuous dose is administered no later than about 4 hours after the first dose.

12. A method as in claim 10 wherein the terminal complement inhibitor compound is an antibody that directly or indirectly reduces the conversion of complement component C5 into complement components C5a and C5b.

13. A method as in claim 12 wherein the complement inhibitor specifically binds to a component forming the C5b-9 complex.

14. A method as in claim 13 wherein the complement inhibitor is h5G1.1-scFv.

15. A method as in claim 14 wherein the second dose is administered over a period of about 24 hours.

16. A method as in claim 15 wherein the first dose is administered over a period of about 10 minutes prior to graft.

17. A method as in claim 16 wherein the first dose is about 2 mg/kg.

18. A method as in claim 17 wherein the second dose is administered at 00.05 mg/kg/hr.

19. A method of reducing sepsis or cardiogenic shock in subjects having coronary artery bypass graft comprising: administering to the subject a first bolus dosage of about 2 mg/kg of pexelizumab about ten minutes prior to anesthesia or the graft; and subsequently administering a second continuous dose of about 1.2 mg/kg pexelizumab intravenously over a period of about 24 hours.

20. A method of reducing sepsis or cardiogenic shock in subjects having an acute myocardial infarction comprising: administering to the subject a first bolus dosage of about 2 mg/kg of pexelizumab in a period selected from the group consisting of not more than 24 hours after the infarction, prior to, during or immediately following a primary percutaneous coronary intervention; and subsequently administering a second continuous dose of about 1.2 mg/kg pexelizumab intravenously over a period of about 24 hours.
